(19) United States
(12) Patent Application Publication
(10) Pub. No.: US 2024/0043626 A1
(43) Pub. Date: Feb. 8, 2024

(54) METHOD FOR THE SELECTIVE CLEAVAGE OF A COMPOUND COMPRISING AN AROMATIC RING AND A C-O-C LINKAGE

(71) Applicants: RHODIA OPERATIONS, Lyon (FR); LE CENTRE NATIONAL DE LA RECHERCHE SCIENTIFIQUE, Paris (FR); UNIVERSITE DE LILLE, Lille (FR); CENTRALE LILLE INSTITUT, Villeneuve-d'Ascq (FR)

(72) Inventors: Dan Wu, Paris (FR); Vitaly Ordomsky, Paris (FR); Andrei Khodakov, Paris (FR); Stephane Streiff, Aubervilliers (FR); Jianxia Zheng, Shanghai (CN)

(73) Assignees: RHODIA OPERATIONS, Lyon (FR); LE CENTRE NATIONAL DE LA RECHERCHE SCIENTIFIQUE, Paris (FR); UNIVERSITE DE LILLE, Lille (FR); CENTRALE LILLE INSTITUT, Villeneuve-d'Ascq (FR)

(21) Appl. No.: 18/257,549

(22) PCT Filed: Dec. 14, 2020
(86) PCT No.: PCT/CN2020/136075

§ 371 Date: Jun. 14, 2023

(51) Int. Cl. C08H 7/00; B01J 23/46; B01J 21/18; B01J 27/13; B01J 21/08; B01J 23/44; C07C 37/54; C07C 1/22

(52) U.S. Cl. CPC C08H 6/00; B01J 23/462; B01J 21/18; B01J 27/13; B01J 21/08; B01J 23/44; C07C 37/54; C07C 1/22

(57) ABSTRACT

A method for the selective cleavage of a compound comprising an aromatic ring and a C—O—C linkage in the presence of a heterogeneous catalyst is provided. The heterogenous catalyst may be a supported noble metal catalyst doped with a halogen selected from the group consisting of chlorine and bromine. By using this method, it is possible to increase the selectivity and/or yield (preferably both) of aromatic compounds.

### TECHNICAL FIELD

The present invention relates to a method for the selective cleavage of a compound comprising an aromatic ring and a C—O—C linkage in the presence of a heterogeneous catalyst.

### BACKGROUND

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of common general knowledge in the field.

Selective hydrogenolysis of the aromatic carbon-oxygen (C—O) bonds in aryl ethers is important for the generation of fuels and chemical feedstocks from biomass and for the liquefaction of coal. It is highly challenging because of their relatively high bond dissociation energies and the competition with alternative hydrogenation reactions.

Due to increased interest in the valorization of the lignin component of biomass, an abundant renewable polymer comprising aromatic units held together by various types of C—O bonds, the need for efficient and selective hydrogenolysis catalysts is essential for biomass valorisation.

Science 2011, 332 (6028), 439-443 reports hydrogenolyses of aromatic C—O bonds in alkyl aryl and diaryl ethers that form exclusively arenes and alcohols. This process is catalyzed by a soluble nickel carbene complex. Benzene and phenol are produced from diphenyl ether without further hydrogenation under very mild conditions (1 bar H₂, 80˜120° C.). However, regardless of the separation of homogeneous catalysts, the using of base additives surfers from purification problem and base waste.

Inorganic Chemistry Communications (2012), 24, 11-15 discloses a homogeneous halogen-containing Ru catalysts. The authors attempted to do the hydrogenolysis of lignin but did not get the conversion of lignin or lignin model compounds.

The using of heterogeneous catalysts in the hydrogenolysis of aromatic C—O bonds is widely reported. For example, ACS Catal. 2019, 9, 4054-4064 reports an in-depth experimental study on the mechanism of Ru/C catalysed hydogenolysis lignin. However, it is necessary to operate at high temperature (>160° C.) and pressure (20 bar H₂), which always leads to aromatic ring saturation.

J Am Chem Soc 2012, 134 (50), 20226-20235 teaches a heterogeneous nickel catalyst for the selective hydrogenolyis of aryl ethers to arenes and alcohols. However, tBuONa must be used in this reaction system. tBuONa a strong basic compound which will introduce problems such as corrosion of the reactor, purification of the products and alkaline waste handling.

Chem Sci 2018, 9 (25), 5530-5535 reports that bimetallic Ru—Ni and Rh—Ni nanocatalysts coated with a phase transfer agent efficiently cleave aryl ether C—O linkages in water in the presence of hydrogen. The authors tested bimetallic Ru—Ni and Rh—Ni catalyst with various Ni ratios for three different lignin model compounds (1-phenoxy-2-phenylethane, benzyl phenyl ether, and diphenyl ether). However, the hydrogenation of the aromatic rings is still inevitable regarding diphenyl ether hydrogenolysis.

ACS Catal. 2018, 8, 11174-11183 reports an efficient H₂-assisted C—O bond cleavage of diphenyl ether in aqueous phase over ultrasmall RuPd bimetallic nanoparticles (NPs) supported on amine-rich silica hollow nanospheres (NH₂—SiO₂). With the reaction time increase the selectivity to benzene and phenol continuously decreased to even zero when diphenyl ether was fully converted.

Hence, it exists a need to provide an improved method for the selective cleavage of a compound comprising an aromatic ring and a C—O-C linkage with increased selectivity and/or yield towards aromatic compounds.

### SUMMARY OF THE INVENTION

An object of the present invention is to increase the selectivity and/or yield (preferably both) in aromatic compounds, typically benzene and phenol, of a method of cleaving a C—O bond in a compound comprising an aromatic ring and a C—O-C linkage, comprising contacting this compound with a hydrogen source in the presence of a supported noble metal catalyst.

Thus, according to a first aspect, the present invention provides a method of cleaving a C—O bond in a compound, comprising contacting the compound with a hydrogen source in the presence of a supported noble metal catalyst doped with a halogen selected from the group consisting of chlorine and bromine, wherein the compound comprises an aromatic ring and a C—O—C linkage, thereby cleaving the C—O bond in the C—O—C linkage.

According to a second aspect, the present invention provides a mixture comprising:
i. a compound comprising an aromatic ring and a C—O—C linkage;
ii. a supported noble metal catalyst doped with a halogen selected from the group consisting of chlorine and bromine;
iii. a hydrogen source;
iv. optionally a solvent;
v. optionally a zeolite having LTA, FAU, BEA, MFI or MOR framework.

Other subjects and characteristics, aspects and advantages of the present invention will emerge even more clearly on reading the detailed description and the examples that follow.

### BRIEF DESCRIPTION OF DRAWINGS

FIG. 1 illustrates the conversion of diphenyl ether (hereinafter “DPE”) and selectivity to benzene, phenol and mono-aromatics when Br—Ru/C was used as the catalyst;

FIG. 2 illustrates the conversion of DPE and selectivity to benzene, phenol and mono-aromatics when Ru/C was used as the catalyst;

FIG. 3 illustrates the evolution of conversion of DPE and yield to different products with the reaction time when Br—Ru/C was used as the catalyst;

FIG. 4 illustrates the stability test of Br—Ru/C catalyst (Conversion of DPE);

FIG. 5 illustrates the stability test of Br—Ru/C catalyst (Selectivity to different products);

FIG. 6 illustrates the conversion of benzyl phenyl ether (hereinafter “BPE”) and selectivity to various products when Br—Ru/C and Ru/C were used as the catalysts.

FIG. 7 illustrates the conversion of dibenzyl ether (hereinafter “DBE”) and selectivity to various products when Br—Ru/C(with and without NaA zeolite) and Ru/C were used as the catalyst.

### DEFINITIONS

Throughout the description, including the claims, the term “comprising one” should be understood as being synonymous with the term “comprising at least one”, unless otherwise specified, and “between” should be understood as being inclusive of the limits.

As used herein, the terminology “(Cₙ-Cₘ)” in reference to an organic group, wherein n and m are both integers, indicates that the group may contain from n carbon atoms to m carbon atoms per group.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

It is specified that, in the continuation of the description, unless otherwise indicated, the values at the limits are included in the ranges of values which are given.

Ratios, concentrations, amounts, and other numerical data may be presented herein in a range format. It is to be understood that such a range format is used merely for convenience and brevity and should be interpreted flexibly to include not only the numerical values explicitly recited as the limits of the range, but also all the individual numerical values or sub-ranges encompassed within that range as if each numerical value or sub-range is explicitly recited.

### DETAILS OF THE INVENTION

Compound Comprising an Aromatic Ring and a C—O—C Linkage

It shall be understood by the skilled person that any one of C—O bonds in the C—O—C linkage can be cleaved by the method according to the present invention.

It shall be understood by the skilled person that the aromatic ring is present in the compound by connecting an aromatic hydrocarbon radical, notably aryl or arenediyl to atom(s), such as carbon or oxygen atom(s), which is contained in the compound.

By “aryl” is meant a monovalent radical obtained by the removal of one hydrogen atom attached to one carbon atom contained in an aromatic ring of an arene, including, but not limited to, phenyl, biphenyl, naphthyl, benzyl, and the like. The aryl includes substituted or unsubstituted aryls. The aryl can have one, two, three, four, or five substituents independently selected from the group consisting of: alkyl, alkenyl, alkynyl, alkoxy, alkylated amino, carboxyl, ester, cyano, nitro and halogen.

By “arenediyl” is meant a bivalent radical obtained by the removal of one hydrogen atom attached to each of two carbon atoms contained in an aromatic ring of an arene, including, but not limited to phenylene. The arenediyl includes substituted or unsubstituted arenediyls. The arenediyl group can have one, two, three or four substituents independently selected from the group consisting of: alkyl, alkenyl, alkynyl, alkoxy, alkylated amino, carboxyl, ester, cyano, nitro and halogen.

Preferably, the aryl is a substituted or unsubstituted phenyl.

By “atom” is meant to include a chemical element, as well as ionic forms thereof. For example, an atom of magnesium is meant to include Mg⁰, as well as ionic forms (e.g., cationic forms, such as Mg²⁺).

In some embodiments, the compound comprising an aromatic ring and a C—O—C linkage may notably be a compound comprising an ether linkage, which belongs to a class of ether linkages that contain an oxygen atom directly connected to at least one aryl or arenediyl.

For example, the compound may comprise an ether linkage, which belongs to a class of ether linkages that contain an oxygen atom directly connected to one alkanediyl, and one aryl or one arenediyl. Non-limiting examples can be a lignin model compound having general formula(I).

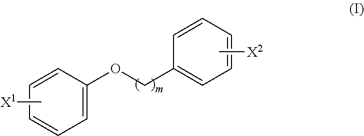

wherein:
alkanediyl is connected to an aryl or an arenediyl;
X¹ and X², independently from one another, are selected from the group consisting of hydrogen, alkyl, alkenyl, alkynyl, alkoxy, alkylated amino, carboxyl, ester, cyano, nitro and halogen and preferably selected from the group consisting of hydrogen, a linear or branched C₁-C₁₂ alkyl, a C₄-C₁₂ cycloalkyl and an aryl;
m is an integer from 1 to 10.

By “alkanediyl” is meant a bivalent radical obtained by the removal of two hydrogen atoms attached to one or two carbon atom(s) of an alkane. The alkanediyl includes substituted or unsubstituted alkanediyls.

The compound having general formula(I) may notably be (benzyloxy)benzene and 1-methyl-4-((4-methylbenzyl)oxy)benzene or phenethoxybenzene and 1-methyl-4-(4-methylphenethoxy)benzene.

For example, the compound comprises an ether linkage, which belongs to a class of ether linkages that contain an oxygen atom directly connected to two aryls or arenediyls. Non-limiting examples can be a lignin model compound having general formula(II) and poly(aryl ether ketone) (PAEK).

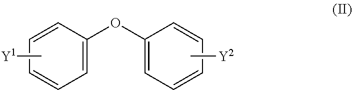

wherein Y¹ and Y² have the same meanings as X¹ and X².

The compound having general formula(II) may notably be diphenyl ether and 4,4′-oxybis(methylbenzene).

As used herein, a poly(aryl ether ketone) (PAEK) denotes any polymer comprising recurring units (R_{PAEK}) comprising a Ar′ —C(═O)—Ar* group, where Ar′ and Ar*, equal to or different from each other, are aromatic groups, the mol. % being based on the total number of moles of recurring units in the polymer. The recurring units (R_{PAEK}) are selected from the group consisting of units of formulas (J-A) to (J-E) below:

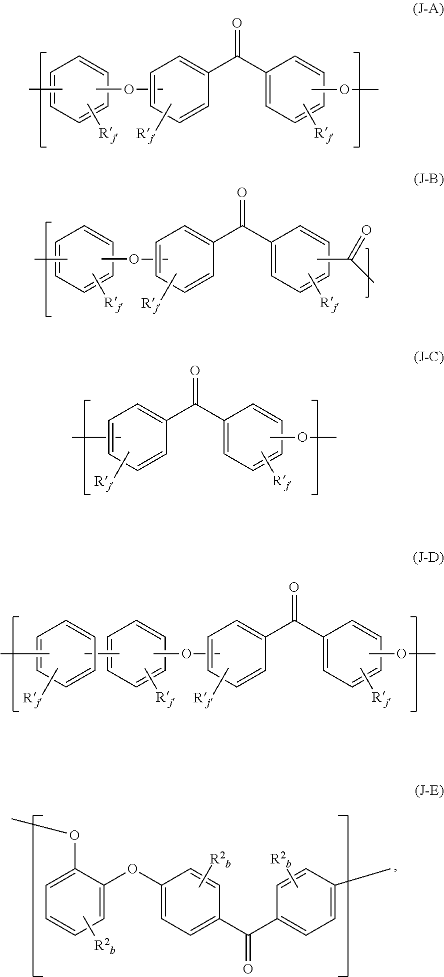

wherein
R′ and R², at each location, is independently selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine and quaternary ammonium; and
j′ and b, are independently zero or an integer ranging from 1 to 4.

In recurring unit (R_{PAEK}), the respective phenylene moieties may independently have 1,2-, 1,4- or 1,3-linkages to the other moieties different from R′ in the recurring unit (R_{PAEK}). Preferably, the phenylene moieties have 1,3- or 1,4- linkages, more preferably they have a 1,4-linkage.

In recurring units (R_{PAEK}), j′ is preferably at each location zero so that the phenylene moieties have no other substituents than those linking the main chain of the polymer.

According to an embodiment, the PAEK is a poly(ether ether ketone) (PEEK).

As used herein, a poly(ether ether ketone) (PEEK) denotes any polymer comprising recurring units (R_{PEEK}) of formula (J-A), based on the total number of moles of recurring units in the polymer:

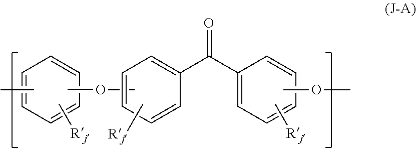

wherein
R′, at each location, is independently selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine and quaternary ammonium; and
j′, for each R′, is independently zero or an integer ranging from 1 to 4 (for example 1, 2, 3 or 4).

According to formula (J-A), each aromatic cycle of the recurring unit (R_{PEEK}) may contain from 1 to 4 radical groups R′. When j′ is 0, the corresponding aromatic cycle does not contain any radical group R′.

Each phenylene moiety of the recurring unit (R_{PEEK}) may, independently from one another, have a 1,2-, a 1,3- or a 1,4-linkage to the other phenylene moieties. According to an embodiment, each phenylene moiety of the recurring unit (R_{PEEK}), independently from one another, has a 1,3- or a 1,4-linkage to the other phenylene moieties. According to another embodiment yet, each phenylene moiety of the recurring unit (R_{PEEK}) has a 1,4-linkage to the other phenylene moieties.

According to an embodiment, R′ is, at each location in formula (J-A) above, independently selected from the group consisting of a C1-C12 moiety, optionally comprising one or more than one heteroatoms; sulfonic acid and sulfonate groups; phosphonic acid and phosphonate groups; amine and quaternary ammonium groups.

According to an embodiment, j′ is zero for each R′. In other words, according to this embodiment, the recurring units (R_{PEEK}) are according to formula (I′ -A):

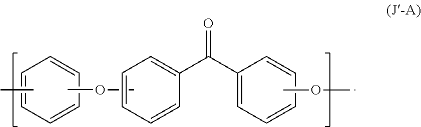

According to another embodiment of the present disclosure, a poly(ether ether ketone) (PEEK) denotes any polymer comprising at least 10 mol. % of the recurring units are recurring units (R_{PEEK}) of formula (J-A″):

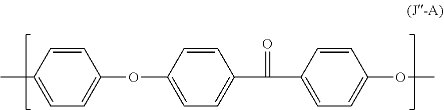

the mol. % being based on the total number of moles of recurring units in the polymer.

According to an embodiment of the present disclosure, at least 10 mol. % (based on the total number of moles of recurring units in the polymer), at least 20 mol. %, at least 30 mol. %, at least 40 mol. %, at least 50 mol. %, at least 60 mol. % , at least 70 mol. %, at least 80 mol. %, at least 90 mol. %, at least 95 mol. %, at least 99 mol. % or all of the recurring units in the PEEK are recurring units (R_{PEEK}) of formulas (J-A), (J′-A) and/or (J″-A).

The PEEK polymer can therefore be a homopolymer or a copolymer. If the PEEK polymer is a copolymer, it can be a random, alternate or block copolymer.

When the PEEK is a copolymer, it can be made of recurring units (R*_{PEEK}), different from and in addition to recurring units (R_{PEEK}).

According to one embodiment, the PAEK is a copolymer of recurring units (R_{PEEK}) as described above and recurring units (R*_{PEEK}) of formula (J-D):

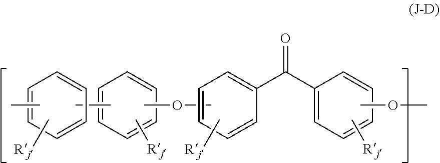

wherein
R′, at each location, is independently selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine and quaternary ammonium; and
j′, for each R′, is independently zero or an integer ranging from 1 to 4.

According to formula (J-D), each aromatic cycle of the recurring unit (R*_{PEEK}) may contain from 1 to 4 radical groups R′. When j′ is 0, the corresponding aromatic cycle does not contain any radical group R′.

According to an embodiment, R′ is, at each location in formula (J-D) above, independently selected from the group consisting of a C1-C12 moiety, optionally comprising one or more than one heteroatoms; sulfonic acid and sulfonate groups; phosphonic acid and phosphonate groups; amine and quaternary ammonium groups.

According to an embodiment, j′ is zero for each R′. In other words, according to this embodiment, the recurring units (R*_{PEEK}) are according to formula (J′-D):

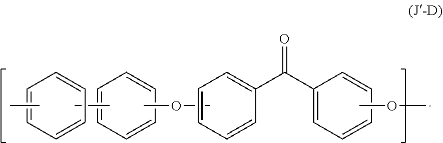

According to another embodiment of the present disclosure, the recurring units (R*_{PEEK}) are according to formula (J″-D):

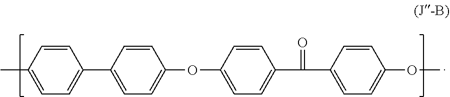

According to an embodiment of the present disclosure, less than 90 mol. % (based on the total number of moles of recurring units in the polymer), less than 80 mol. %, less than 70 mol. %, less than 60 mol. %, less than 50 mol. %, less than 40 mol. %, less than 30 mol. %, less than 20 mol. %, less than 10 mol. %, less than 5 mol. %, less than 1 mol. % or all of the recurring units in the PEEK are recurring units (R*_{PEEK}) of formulas (J-D), (J′-D), and/or (J″-D).

According to an embodiment, the PEEK polymer is a PEEK-PEDEK copolymer. As used herein, a PEEK-PEDEK copolymer denotes a polymer comprising recurring units (R_{PEEK}) of formula (J-A), (J′-A) and/or (J″-A) and recurring units (R*_{PEEK}) of formulas (J-D), (J′-D) or (J″-D) (also called hereby recurring units (R_{PEDEK})). The PEEK-PEDEK copolymer may include relative molar proportions of recurring units (R_{PEEK}/R_{PEDEK}) ranging from 95/5 to 5/95, from 90/10 to 10/90, or from 85/15 to 15/85. The sum of recurring units (R_{PEEK}) and (R_{PEDEK}) can for example represent at least 60 mol. %, 70 mol. %, 80 mol. %, 90 mol. %, 95 mol. %, 99 mol. %, of recurring units in the PEEK copolymer. The sum of recurring units (R_{PEEK}) and (R_{PEDEK}) can also represent 100 mol. %, of recurring units in the PEEK copolymer.

According to one embodiment, the PAEK is a copolymer of recurring units (R_{PEEK}) as described above and recurring units (R_{PEEK}) of formula (J-E):

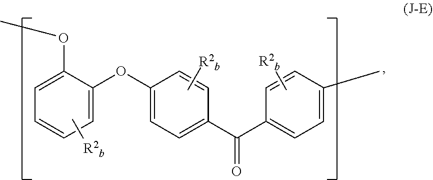

wherein
R², at each location, is independently selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine and quaternary ammonium; and
b, for each R², is independently zero or an integer ranging from 1 to 4.

According to formula (J-E), each aromatic cycle of the recurring unit (R_{PEEK}) may contain from 1 to 4 radical groups R². When b is 0, the corresponding aromatic cycle does not contain any radical group R².

According to an embodiment, R² is, at each location in formula (J-E) above, independently selected from the group consisting of a C1-C12 moiety, optionally comprising one or more than one heteroatoms; sulfonic acid and sulfonate groups; phosphonic acid and phosphonate groups; amine and quaternary ammonium groups.

According to an embodiment, b is zero for each R². In other words, according to this embodiment, the recurring units (R*_{PEEK}) are according to formula (J′-E):

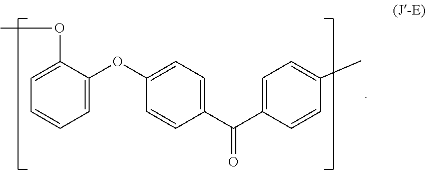

According to an embodiment of the present disclosure, less than 90 mol. % (based on the total number of moles of recurring units in the polymer), less than 80 mol. %, less than 70 mol. %, less than 60 mol. %, less than 50 mol. %, less than 40 mol. %, less than 30 mol. %, less than 20 mol. %, less than 10 mol. %, less than 5 mol. %, less than 1 mol. % or all of the recurring units in the PEEK are recurring units (R_{PEEK}) of formulas (J-E) and/or (J′-E).

In some embodiments, the PAEK is a PEEK-PEoEK copolymer, that-is-to-say a copolymer comprsing PEEK recurring units and PEoEK recurring units. As used herein, a PEEK-PEoEK copolymer denotes a polymer comprising recurring units (R_{PEEK}) of formula (J-A), (J′-A) and/or (J″-A) and recurring units (R_{PEEK}) of formulas (J-E) and/or (J′-E) (also called hereby recurring units (R_{PEoEK}). The PEEK-PEoEK copolymer may additionally comprise recurring units different from recurring units (R_{PEEK}) and (R_{PEoEK}), as above detailed. In such case, the amount of these repeat units can be comprised between 0.1 and less than 50 mol. %, preferably less than 10 mol. %, more preferably less than 5 mol. %, most preferably less than 2 mol. %, with respect to the total number of moles of recurring units of PEEK-PEoEK copolymer. Recurring units R_{PEEK} and R_{PEoEK} are present in the PEEK-PEoEK copolymer in a R_{PEEK}/R_{PEoEK} molar ratio ranging from 95/5 to 5/95. Preferably, the PEEK-PEoEK copolymers are those comprising a majority of R_{PEEK} units, that-is-to-say copolymers in which the R_{PEEK}/R_{PEoEK} molar ratio ranges from 95/5 to more than 50/50, even more preferably from 95/5 to 60/40, still more preferably from 90/10 to 65/35, most preferably 85/15 to 70/30.

PEEK is commercially available as KetaSpire® PEEK from Solvay Specialty Polymers USA, LLC.

PEEK can be prepared by any method known in the art. It can for example result from the condensation of 4,4′-difluorobenzophenone and hydroquinone in presence of a base. The reactor of monomer units takes place through a nucleophilic aromatic substitution. The molecular weight (for example the weight average molecular weight Mw) can be adjusting the monomers molar ratio and measuring the yield of polymerisation (e.g. measure of the torque of the impeller that stirs the reaction mixture).

According to one embodiment of the present disclosure, the PEEK polymer has a weight average molecular weight (Mw) ranging from 75,000 to 100,000 g/mol, for example from 77,000 to 98,000 g/mol, from 79,000 to 96,000 g/mol, from 81,000 to 95,000 g/mol, or from 85,000 to 94,500 g/mol (as determined by gel permeation chromatography (GPC) using phenol and trichlorobenzene (1:1) at 160° C., with polystyrene standards).

In another embodiment, the PAEK is a poly(ether ketone ketone) (PEKK).

As used herein, a poly(ether ketone ketone) (PEKK) denotes a polymer comprising more than 50 mol. % of the recurring units of formulas (J-B₁) and (J-B₂), and at least one recurring unit of each, the mol. % being based on the total number of moles of recurring units in the polymer:

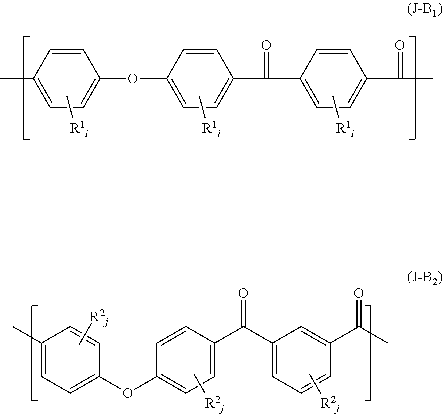

wherein
R¹ and R², at each instance, is independently selected from the group consisting of an alkyl, an alkenyl, an alkynyl, an aryl, an ether, a thioether, a carboxylic acid, an ester, an amide, an imide, an alkali or alkaline earth metal sulfonate, an alkyl sulfonate, an alkali or alkaline earth metal phosphonate, an alkyl phosphonate, an amine, and a quaternary ammonium; and
i and j, at each instance, is an independently selected integer ranging from 0 to 4.

According to an embodiment, R¹ and R² are, at each location in formula (J-B₂) and (J-B₁) above, independently selected from the group consisting of a C1-C12 moiety, optionally comprising one or more than one heteroatoms; sulfonic acid and sulfonate groups; phosphonic acid and phosphonate groups; amine and quaternary ammonium groups.

According to another embodiment, i and j are zero for each R¹ and R² group. According to this embodiment, the PEKK polymer comprises at least 50 mol. % of recurring units of formulas (J′-B₁) and (J′-B₂), the mol. % being based on the total number of moles of recurring units in the polymer:

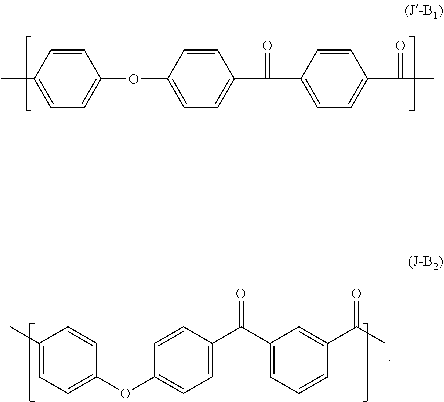

According to an embodiment of the present disclosure, at least 55 mol. %, at least 60 mol. %, at least 70 mol. %, at least 80 mol. %, at least 90 mol. %, at least 95 mol. %, at least 99 mol. % or all of the recurring units in the PEKK are recurring units of formulas (J-B₁) and (J-B₂).

According to an embodiment of the present disclosure, in the PEKK polymer, the molar ratio of recurring units (J-B₂) or/and (J′-B₂) to recurring units (J-B₁) or/and (J′-B₁) is at least 1:1 to 5.7:1, for example at least 1.2:1 to 4:1, at least 1.4:1 to 3:1 or at least 1.4:1 to 1.86:1.

The PEKK polymer has preferably an inherent viscosity of at least 0.50 deciliters per gram (dL/g), as measured following ASTM D2857 at 30° C. on 0.5 wt./vol. % solutions in concentrated H₂SO₄ (96 wt. % minimum), for example at least 0.60 dL/g or at least 0.65 dL/g and for example at most 1.50 dL/g, at most 1.40 dL/g, or at most 1.30 dL/g.

PEKK is commercially available as NovaSpire® PEKK from Solvay Specialty Polymers USA, LLC.

In some embodiments, the compound comprising an aromatic ring and a C—O—C linkage may be a compound comprising an ether linkage, which belongs to a class of ether linkages that contain an oxygen atom directly connected to two alkanediyls, each of which is connected to an aryl or an arenediyl. Non-limiting examples can be a lignin model compound having general formula (III).

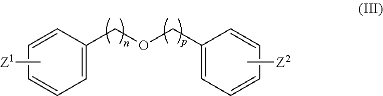

wherein Z¹ and Z² have the same meanings as X¹ and X²; n and p, independently from one another, are integers from 1 to 10.

The compound having general formula (III) may notably be dibenzyl ether and (oxybis(methylene))dibenzene.

In some embodiments, the compound comprising an aromatic ring and a C—O—C linkage is a lignin compound. Lignin compound is a class of aromatic biopolymers, which comprises ether linkages above defined.

Catalyst

As previously expressed, a supported noble metal catalyst doped with a halogen selected from the group consisting of chlorine and bromine is used in the method according to the present invention.

The noble metals are metals that are normally valuable and resistant to corrosion and oxidation in moist air. Preferred noble metal can be selected from the group consisting of rhenium, ruthenium, rhodium, palladium, silver, osmium, iridium, platinum and gold. Ruthenium is most preferable among these noble metals.

Advantageously, the noble metal may be present in amount from 0.5 wt % to 30 wt %, more preferably 2 wt % to 10 wt % in the supported noble metal catalyst, relative to the total weight of the supported noble metal catalyst with a dopant.

The noble metal is normally present in the form of nanoparticles on the support. The average particle size may be from 0.5 to 30 nm and preferably from 1 to 10 nm.

A person skilled in the art will understand how to prepare such a TEM image and determine the particle size based on the magnification. For example, Pd nanoparticles can be characterized by TEM on a JEOL JEM 2100 microscope operated at 200 kV and equipped with Energy Dispersive Spectroscopy (EDS). The particles to be measured refer to the projection (2D-representation) of the particles on the micrograph. Before performing the measurements, it is necessary to calibrate the image. Size distribution histograms are then plotted as percent Pd nanoparticles versus Pd diameter on the basis of the size measurements obtained from an image processing program, such as ImageJ. The number average is obtained by weighted average method. The measurement should be made on a sufficiently high number of particles, for example at least 25 particles, preferably at least 100 particles, more preferably at least 300 particles, still more preferably at least 500 particles.

The support is not particularly limited as long as its presence does not prevent the cleavage reaction.

The support can be a metal oxide selected from the group consisting of aluminum oxide (Al₂O₃), silicon dioxide (SiO₂), titanium oxide (TiO₂), zirconium dioxide (ZrO₂), calcium oxide (CaO), magnesium oxide (MgO), lanthanum oxide (La₂O₃), niobium dioxide (NbO₂), cerium oxide (CeO₂) and mixtures thereof. Preferably, said support is silicon dioxide.

The support can be a zeolite. Zeolites are substances having a crystalline structure and a unique ability to change ions. People skilled in the art can easily understand how to obtain those zeolites by preparation method reported, such as zeolite L is described in U.S. Pat. No. 4,503,023 or commercial purchase, such as ZSM available from ZEOLYST.

The support can also be Kieselguhr, clay or carbon and preferably carbon.

The supported catalysts used in the method according to the present invention include those commercially available, such as Ru/C from Johnson Matthey.

The halogen, acting as a dopant, may preferably be Br.

The halogen source can be organic or inorganic halogen source.

Examples of halogen source can be:
Halobenzene, such as chlorobenzene and bromobenzene;
Elemental halogen, such as Cl₂, Br₂;
Haloalkane, such as 1-bromohexadecane;
Ammonium halide, such as NH₄Cl and NH₄Br;
Alkali metal halide, such as KCl, KBr, NaCl and NaBr.

Advantageously, the halogen may be present in amount from 0.05 wt % to 5 wt %, more preferably 0.5 wt % to 2 wt % in the supported noble metal catalyst, relative to the total weight of the supported noble metal catalyst with a dopant.

The loading of halogens is analyzed by Energy Dispersive X-ray Spectroscopy (EDS). For example, a JEOL Silicon Drift Detector (DrySD60GV, sensor size 60 mm²) with a solid angle of approximately 0.6 srad has been used for halogens analysis.

Advantageously, the weight ratio of noble metal to halogen is from 1 to 60 and preferably from 5 to 20.

The catalyst can be prepared by some well-known ways, such as described in the patent WO 2020/000170 A1. In a typical method, a supported noble metal catalyst, a halogen source and a solvent is mixed in the presence of H₂ under proper reaction temperature for proper time. After reaction, the catalyst was separated, washed and dried.

The solvents used for preparing the catalyst are not particularly limited. The solvent may be selected from the group consisting of alkane, alkene, arene, halogenated-hydrocarbon, ether, ester, ketone, alcohol, or any combination thereof. Exemplary solvents include methanol, ethanol, isopropanol, acetone, tetrahydrofuran, and any combination thereof.

Advantageously, the solvent is substantially free or completely free of water.

In some embodiments, the solvent is substantially free of water.

As used herein, the term “substantially free of water” when used with reference to the solvent means that the solvent comprises no more than 0.5 wt. %, preferably no more than 0.2 wt. % of water, based on the total weight of the solvent.

In some embodiments, the solvent is completely free of water.

As used herein, the term “completely free of water” when used with reference to the solvent means that the solvent comprises no water at all.

The reaction time for preparing the catalyst may be from 1 to 24 h and preferably from 2 to 10 h.

The reaction for preparing the catalyst may be carried under a H₂ pressure from 1 and 50 bars, preferably between 2 to 8 bars and more preferably 3 to 7 bars.

Advantageously, the weight ratio of the compound comprising an aromatic ring and a C—O—C linkage to the catalyst may be from 1:1 to 100:1 and preferably from 2:1 to 10:1.

Hydrogen Source

The hydrogen source can be H₂, NaBH₄ or LiAlH₄ and preferably H₂. When H₂ is used, the cleavage reaction may be carried under a H₂ pressure from 1 and 50 bars, preferably 2 to 8 bars and more preferably 3 to 7 bars.

Solvent

The solvents used for the cleavage reaction are not particularly limited. Any solvent has good solubility for the compound comprising an aromatic ring and a C—O—C linkage can be used. The solvent may be selected from the group consisting of alkane, alkene, arene, halogenated-hydrocarbon, ether, ester, ketone, alcohol, or any combination thereof. Exemplary solvents include methanol, ethanol, isopropanol, acetone, tetrahydrofuran, and any combination thereof.

Preferably, the weight ratio of the compound comprising an aromatic ring and a C—O—C linkage to the solvent may be from 0.005:1 to 1:1 and preferably from 0.02:1 to 0.1:1.

Zeolite

Advantageously, the cleavage reaction may be carried out in the presence of a zeolite having LTA, FAU, BEA, MFI or MOR framework and preferably LTA framework, such as NaA zeolite.

The weight ratio of the zeolite to the compound comprising an aromatic ring and a C—O—C linkage may be from 0.01:1 to 50:1 and preferably from 1:1 to 10:1.

Reaction Temperature

The reaction temperature of the cleavage reaction may be from 80 to 250° C. and preferably from 110 to 130° C.

Reaction Time

The reaction time of the cleavage reaction may be from 1 to 24 h, preferably from 3 to 10 h, and more preferably 4 to 7 h.

Compared with the methods previously reported for this type of reaction, the method according to the present invention has several advantages, including:
a) higher selectivity and/or yield(preferably both) towards aromatic compounds;
b) mild operating conditions, such as lower reaction temperature and H₂ gas pressure;
c) the catalyst used therein can be reused several times (at least 3 times) without significant losses in the catalytic efficiency.

In some preferred embodiments, such as when Br—Ru/C is used, it is possible to selectively cleave C—O bond in the C—O—C linkage without or almost without hydrogenation of aromatic rings.

By “almost without” is meant that less than 20 mole percentage and preferably less than 5 mole percentage of aromatic rings in the compound is subject to further hydrogenation.

The present invention provides a mixture comprising:
i. a compound comprising an aromatic ring and a C—O—C linkage;
ii. a supported noble metal catalyst doped with a halogen selected from the group consisting of chlorine and bromine;
iii. a hydrogen source;
iv. optionally a solvent;
v. optionally a zeolite having LTA, FAU, BEA, MFI or MOR framework.

The compound comprising an aromatic ring and a C—O—C linkage, the catalyst, the hydrogen source, the solvent and the zeolite are as defined above.

The following examples are included to illustrate embodiments of the invention. Needless to say, the invention is not limited to described examples.

Experimental Part

Materials

Commercial 5 wt. % Ru/C, 5 wt. % Ru/SiO₂, and 5 wt. % Pd/C catalyst were purchased from Johnson Matthey Chemicals Company. Bromobenzene, chlorobenzene, iodobenzene, methanol, DPE, BPE, DBE, lignin (alkali), and lignosulfonic acid calcium salt were supplied by Sigma-Aldrich company. Lignin (dealkaline) was supplied by TCL chemical company. Air, nitrogen, and hydrogen were supplied by Air Liquide company. Deionized water was obtained from a Millipore system. All chemicals were analytical grade and used as received without further purification.

Catalyst Preparation:

200 mg 5 wt. % Ru/C (or 5 wt. % Ru/SiO₂ or 5 wt. % Pd/C) catalyst, 50 mg bromobenzene, chlorobenzene or iodobenzene, and 5 ml methanol were put together in a 50 ml bath reactor. The reactor was sealed and pressurized with 5 bar of H₂, then heating at 120° C. for 3 hours. After reaction, the catalyst (Cl—Ru/C or Br—Ru/C or I—Ru/C or Br—Ru/SiO₂ or Br—Pd/C) was separated and washed with methanol for 3 times, and dried at 60° C. in the oven overnight. The amount of Br, Cl, I was measured by EDS. The amount of Br is 1.2 wt. % in Br—Ru/C, 1.0 wt% in Br—Ru/SiO₂, 1.3 wt. % in Br—Pd/C. The amount of Cl is 1.3 wt. % in Cl—Ru/C and the amount of I is 1.4 wt. % in I—Ru/C.

Synthesis Procedure of ((1,4-phenylenebis(oxy))bis(4,1-phenylene))bis((4-methoxyphenyl)methanone)

1.26 g (82.2 mmol, 2 equiv.) of p-methoxybenzoic acid and 1.12 g (41.1 mmol, 1 equiv.) of 1,1-diphenoxybenzene were weighed in a 120 mL Schlenk (40.80 g) of Eaton's reagent previously prepared by dissolving 7.7% w/w of P₂O₅ in methanesulfonic acid were introduced. The resulting mixture stirred for 60 hours at room temperature. The medium was then neutralized with a 1 N NaOH solution at 0° C. The precipitate was filtered under vacuum and washed with water. A mass of 1.93 g of a pink sold was obtained with a yield of 88%.

¹H NMR (300 MHz, CDCl₃): δ3.89 (s, 6H), 6.96-6.98 (m, 4H), 7.04-7.06 (m, 4H), 7.13 (s, 4H), 7.78-7.82 (m, 8H).

¹³C NMR (75.5 MHz, CDCl₃): δ55.51 (2 C), 113.57 (4 C), 117.03 (4 C), 121.60 (4 C), 132.17-132.34 (4 C).

IR: 1639 cm⁻¹, 1599 cm⁻¹, 1501 cm⁻¹, 1414 cm⁻¹, 1306 cm⁻¹, 1291 cm⁻¹.

Example 1:

50 mg Br—Ru/C, 100 mg DPE, and 5 g methanol were put together in a 50 ml batch reactor. Then, pressurized 5 bar of H₂, and heated at 120° C. for 6 h. The products were analyzed by GC and GC-MS, with a normalization method for quantity. The main products are mono-aromatics: benzene (Bez), phenol (PhOH) and trace amount of anisole. The selectivities and yields of Bez and PhOH are shown in Table 1. The main by-products are cyclohexane (CHE), cyclohexanol (CHOH), dicyclohexyl ether (CHOCH) and (cyclohexyloxy)-benzene (CHOBez). FIG. 1 shows the conversion of DPE and selectivity to benzene, phenol and mono-aromatics. FIG. 3 shows the evolution of conversion of DPE and yield to different products with the reaction time.

Comparative Example 1:

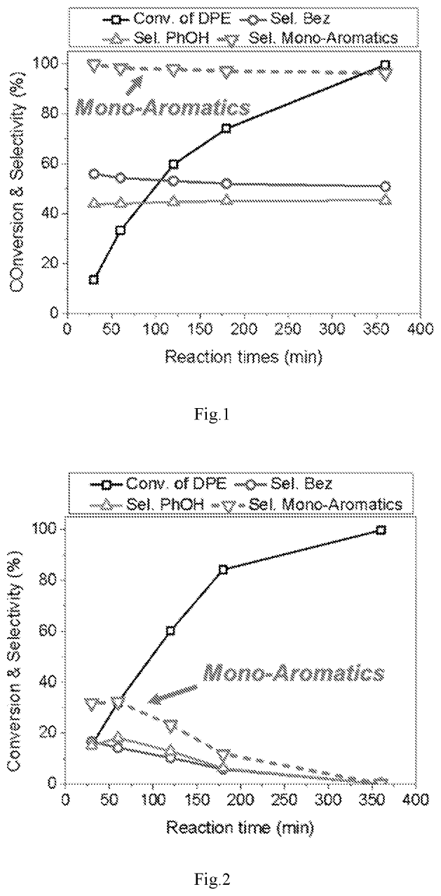

This example was performed in the same way as Example 1 except the catalyst is replaced by 5 wt. % Ru/C. The selectivities and yields of Bez and PhOH are shown in Table 1. FIG. 2 shows the conversion of DPE and selectivity to benzene, phenol and mono-aromatics.

Example 2:

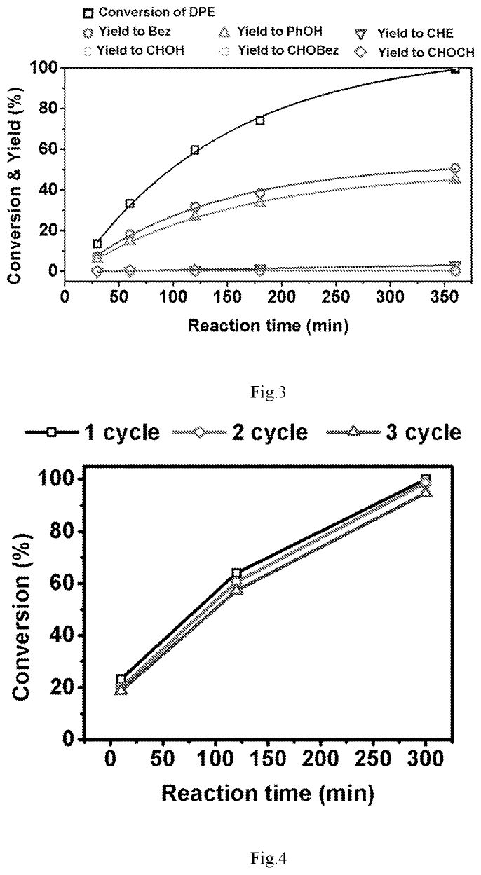

The stability of Br—Ru/C catalyst was tested by hydrogenolysis of DPE at 120° C. and 5 bar of H₂ with 50 mg of Br—Ru/C, 100 mg DPE, and 5 g methanol in three consecutive cycles with intermediate separation of the catalyst. As shown by FIG. 4, the catalyst demonstrates comparable activity in DPE transformation without obvious decrease for 2 and 3 cycles. The selectivity curves in FIG. 5 are very similar for all three cycles with the continuous high selectivity to benzene and phenol. It indicates the same state of the Br—Ru/C catalyst during reaction.

Example 3:

50 mg Cl—Ru/C, 100 mg DPE, and 5 g methanol were put together in a 50 ml batch reactor. Then, pressurized 5 bar of H₂, and heating at 120° C. for 6 h. The products were analyzed by GC and GC-MS, with a normalization method for quantity. The selectivities and yields of Bez and PhOH are shown in Table 1.

Comparative Example 2:

50 mg I-Ru/C, 100 mg DPE, and 5 g methanol were put together in a 50 ml batch reactor. Then, pressurized 5 bar of H₂, and heating at 120° C. for 6 h. The products were analyzed by GC and GC-MS, with a normalization method for quantity. The selectivities and yields of Bez and PhOH are shown in Table 1.

TABLE 1

| | | Selectivity | | | Yield | | |
|---|---|---|---|---|---|---|---|
| Catalyst | Conv. (%) | Bez | PhOH | Bez + PhOH | Bez | PhOH | Bez + PhOH |
| Ru/C | 100 | 0 | 0 | 0 | 0 | 0 | 0 |
| I—Ru/C | 0.8 | 0 | 0 | 0 | 0 | 0 | 0 |
| Cl—Ru/C | 100 | 8.9 | 9.7 | 18.6 | 8.9 | 9.7 | 18.6 |
| Br—Ru/C | 100 | 49.6 | 49.8 | 99.4 | 49.6 | 49.8 | 99.4 |

Example 4:

50 mg Br—Ru/SiO₂, 100 mg DPE, and 5 g methanol were put together in a 50 ml batch reactor. Then, pressurized 5 bar of H₂, and heating at 120° C. for 6 h. The products were analyzed by GC and GC-MS, with a normalization method for quantity. The selectivities and yields of Bez and PhOH are shown in Table 2.

Comparative Example 3:

50 mg 5 wt. % Ru/SiO₂, 100 mg DPE, and 5 g methanol were put together in a 50 ml batch reactor. Then, pressurized 5 bar of H₂, and heating at 120° C. for 6 h. The products were analyzed by GC and GC-MS, with a normalization method for quantity. The selectivities and yields of Bez and PhOH are shown in Table 2.

TABLE 2

| | | Selectivity(%) | | | Yield(%) | | |
|---|---|---|---|---|---|---|---|
| Catalyst | Conv. (%) | Bez | PhOH | Bez + PhOH | Bez | PhOH | Bez + PhOH |
| Ru/SiO₂ | 47.7 | 5.1 | 4.6 | 9.7 | 2.4 | 2.2 | 4.6 |
| Br—Ru/SiO₂ | 18.6 | 48.4 | 47.6 | 96 | 9.0 | 8.9 | 17.9 |

Example 5:

50 mg Br—Pd/C, 100 mg DPE, and 5 g methanol were put together in a 50 ml batch reactor. Then, pressurized 5 bar of H₂, and heating at 120° C. for 6 h. The products were analyzed by GC and GC-MS, with a normalization method for quantity. The selectivities and yields of Bez and PhOH are shown in Table 3.

Comparative Example 4:

50 mg 5 wt. % Pd/C, 100 mg DPE, and 5 g methanol were put together in a 50 ml batch reactor. Then, pressurized 5 bar of H₂, and heating at 120° C. for 6 h. The products were analyzed by GC and GC-MS, with a normalization method for quantity. The selectivities and yields of Bez and PhOH are shown in Table 3.

TABLE 3

| | | Selectivity(%) | | | Yield(%) | | |
|---|---|---|---|---|---|---|---|
| Catalyst | Conv. (%) | Bez | PhOH | Bez + PhOH | Bez | PhOH | Bez + PhOH |
| Pd/C | 38.9 | 2.3 | 0.6 | 2.9 | 0.9 | 0.2 | 1.1 |
| Br—Pd/C | 27.3 | 7.2 | 17.4 | 24.6 | 2.0 | 4.8 | 6.8 |

Example 6:

50 mg Br—Ru/C, 100 mg (benzyloxy)benzene (BPE), and 5 g methanol were put together in a 50 ml batch reactor. Then, pressurized 5 bar of H₂, and heating at 120° C. for 3 h. The products were analyzed by GC and GC-MS, with a normalization method for quantity. The products are cyclohexane (CHE), benzene (Bez), methylcyclohexane (MCHE), toluene (TL), cyclohexanol (CHOH), phenol (PhOH), cyclohexylmethanol (CHMOH), benzyl alcohol (BezMOH), (cyclohexylmethoxy)cyclohexane (CHOMCH), ((cyclohexyloxy)methyl)benzene (CHOMBez) and (cyclohexylmethoxy)benzene (BezOMCH) in Scheme 1.

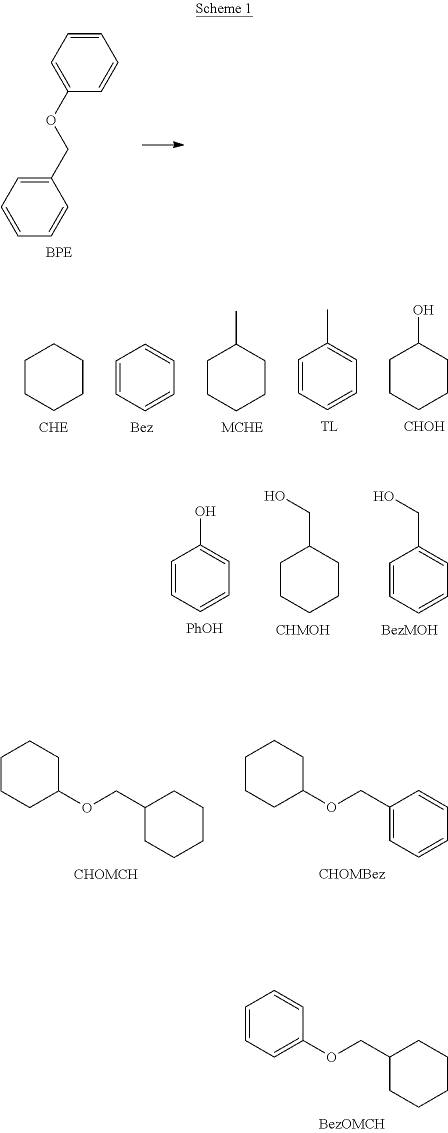

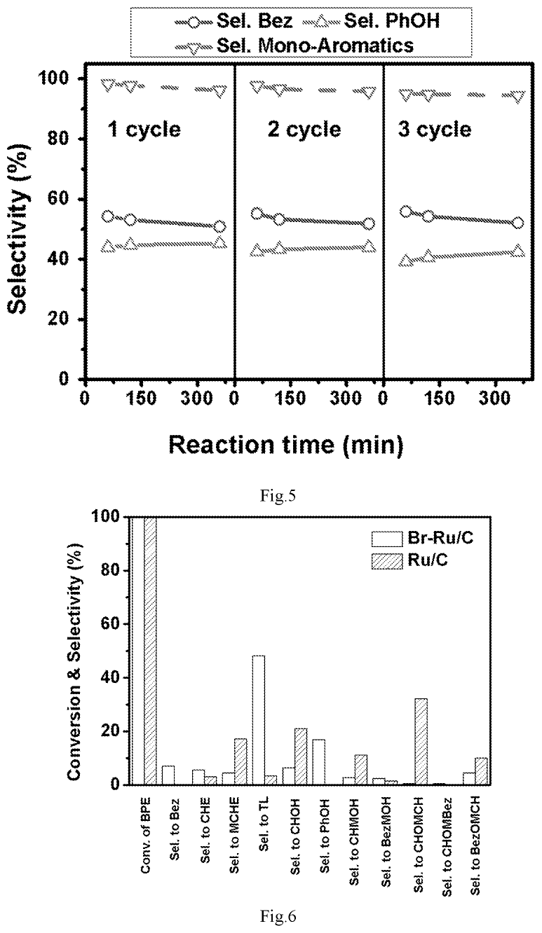

FIG. 6 shows the conversion of BPE and selectivity to various products. When Br—Ru/C was used, the higher selectivity (above 85%) of aromatic products was obtained.

Example 7:

50 mg Br—Ru/C, 100 mg dibenzyl ether (DBE) and 5 g methanol were put together in a 50 ml batch reactor. Then, pressurized 5 bar of H₂, and heating at 120° C. for 6 h. The products were analyzed by GC and GC-MS, with a normalization method for quantity. The products are methyl cyclohexane (MCHE), toluene (TL), cyclohexylmethanol (CHMOH), and (oxybis(methylene))dicyclohexane (CHMOMCH) in Scheme 2.

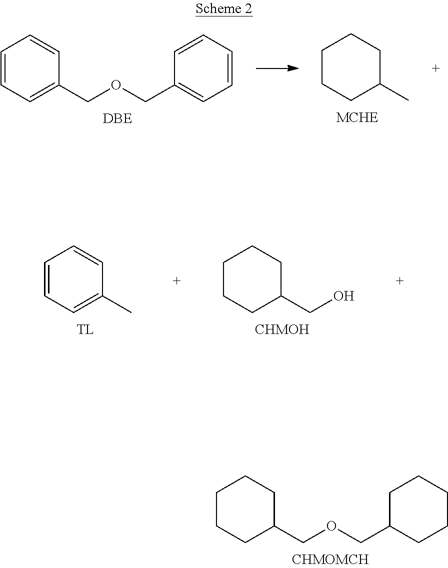

Example 8:

50 mg Br—Ru/C, 100 mg dibenzyl ether (DBE), 5 g methanol and 1 g NaA zeolite were put together in a 50 ml batch reactor. Then, pressurized 5 bar of H₂, and heating at 120° C. for 6 h. The products were analyzed by GC and GC-MS, with a normalization method for quantity. The products are methyl cyclohexane (MCHE), toluene (TL), cyclohexylmethanol (CHMOH), and (oxybis(methylene))dicyclohexane (CHMOMCH) in Scheme 2.

Comparative Example 5:

50 mg Ru/C, 100 mg dibenzyl ether (DBE) and 5 g methanol were put together in a 50 ml batch reactor. Then, pressurized 5 bar of H₂, and heating at 120° C. for 6 h. The products were analyzed by GC and GC-MS, with a normalization method for quantity. The products are methylcyclohexane (MCHE), toluene (TL), cyclohexylmethanol (CHMOH), and (oxybis(methylene))dicyclohexane (CHMOMCH) in Scheme 2.

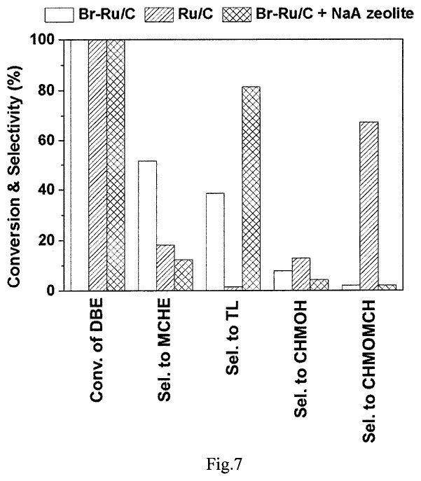

FIG. 7 shows the conversion of DBE and selectivity to various products. When Br—Ru/C was used, the higher selectivity (above 38.5%) of aromatic products was obtained. In the case when the NaA zeolite was used as water scavenger in the reaction mixture, the higher selectivity (above 81.4%) of aromatic products was obtained.

Example 9:

50 mg Br—Ru/C, 100 mg ((1,4-phenylenebis(oxy))bis(4,1-phenylene))bis((4-methoxyphenyl)methanone), and 5 g methanol were put together in a 50 ml batch reactor. Then, pressurized 5 bar of H₂, and heating at 120° C. for 3 h. The products were analyzed by GC and GC-MS, with a normalization method for quantity.

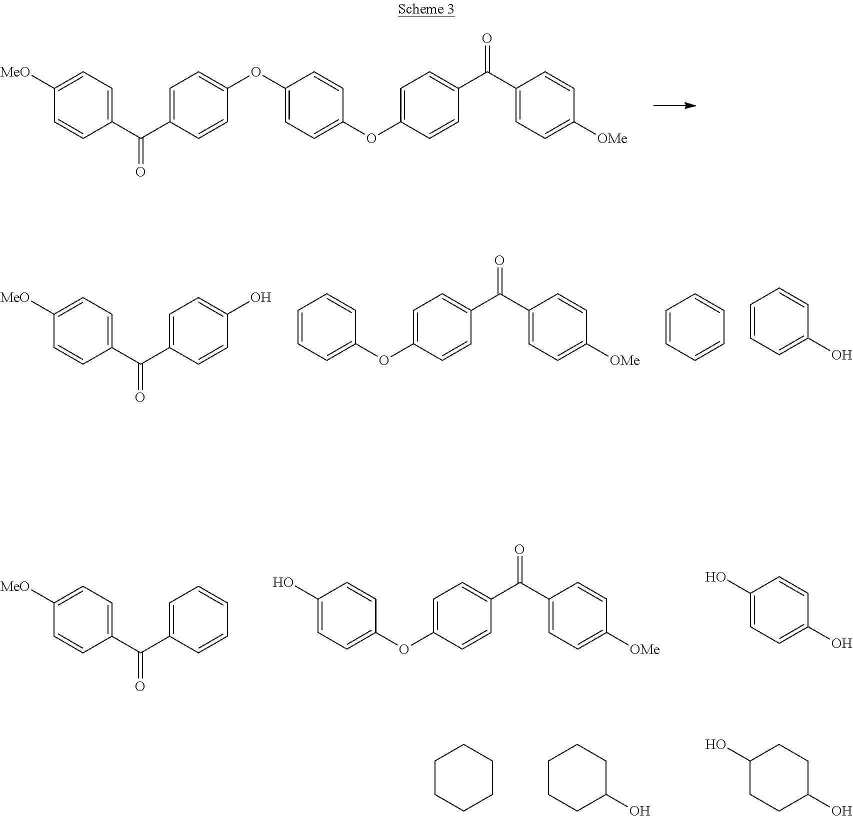

The products are (4-hydroxyphenyl)(4-methoxyphenyl)methanone, (4-methoxyphenyl)(4-phenoxyphenyl)methanone, benzene, phenol, (4-(4-hydroxyphenoxy)phenyl)(4-methoxyphenyl)methanone, (4-methoxyphenyl)-(phenyl)methanone, hydroquinone, cyclohexane, cyclohexanol, cyclohexane-1,4-diol in Scheme 3. It is expected that selectivity and/or yield towards aromatic products will be obtained by this reaction.

Example 10:

50 mg Br—Ru/C, 50 mg lignin (alkali) and 10 g methanol were put together in a 50 ml batch reactor. Then, pressurized 5 bar of H₂, and heating at 180° C. for 6 h. The products were analyzed by GC and GC-MS, with biphenyl as internal standard. It is expected that selectivity and/or yield towards aromatic products will be obtained by this reaction.

Example 11:

50 mg Br—Ru/C, 50 mg lignosulfonic acid calcium salt and 10 g methanol were put together in a 50 ml batch reactor. Then, pressurized 5 bar of H₂, and heating at 180° C. for 6 h. The products were analyzed by GC and GC-MS, with biphenyl as internal standard. It is expected that selectivity and/or yield towards aromatic products will be obtained by this reaction.

Example 12:

50 mg Br—Ru/C, 50 mg 1 lignin (dealkaline) and 10 g methanol were put together in a 50 ml batch reactor. Then, pressurized 5 bar of H₂, and heating at 180° C. for 6 h. The products were analyzed by GC and GC-MS, with biphenyl as internal standard. It is expected that selectivity and/or yield towards aromatic products will be obtained by this reaction.

1. A method of cleaving a C—O bond in a compound, comprising contacting the compound with a hydrogen source in the presence of a supported noble metal catalyst doped with a halogen selected from the group consisting of chlorine and bromine, wherein the compound comprises an aromatic ring and a C—O—C linkage, thereby cleaving the C—O bond in the C—O—C linkage.

2. The method according to claim 1, wherein the compound comprising the aromatic ring and the C—O—C linkage is a compound comprising an ether linkage, which belongs to a class of ether linkages that contain an oxygen atom directly connected to at least one aryl or arenediyl or a class of ether linkages that contain an oxygen atom directly connected to two alkanediyls, each of which is connected to an aryl or an arenediyl.

3. The method according to claim 1, wherein the compound comprising an aromatic ring and a C—O—C linkage is a lignin compound.

4. The method according to claim 2, wherein the compound comprising an aromatic ring and the C—O—C linkage is a compound comprising an ether linkage, which is a class of ether linkages that contain one oxygen atom directly connected to two aryls or arenediyls.

5. The method according to claim 4, wherein the compound comprising the aromatic ring and the C—O—C linkage is a poly(aryl ether ketone) (PAEK) comprising recurring units (R_{PAEK}) which are selected from the group consisting of units of formulas (J-A) to (J-E) below: 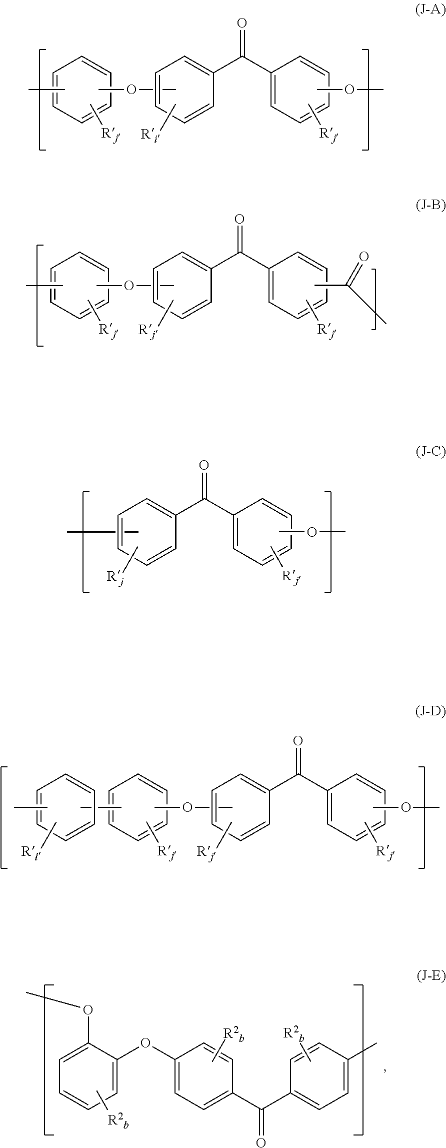
wherein
R′ and R², at each location, is independently selected from the group consisting of halogen, alkyl, alkenyl, alkynyl, aryl, ether, thioether, carboxylic acid, ester, amide, imide, alkali or alkaline earth metal sulfonate, alkyl sulfonate, alkali or alkaline earth metal phosphonate, alkyl phosphonate, amine, quaternary ammonium; and
j′ and b, are independently zero or an integer ranging from 1 to 4.

6. The method according to claim 1, wherein the noble metal is selected from the group consisting of rhenium, ruthenium, rhodium, palladium, silver, osmium, iridium, platinum and gold, and combinations thereof.

7. The method according to claim 1, wherein the noble metal is present in amount from 0.5 wt % to 30 wt % relative to the total weight of the supported noble metal catalyst with a dopant.

8. The method according to claim 1, wherein the halogen is Br.

9. The method according to claim 1, wherein the halogen is present in amount from 0.05 wt % to 5 wt % relative to the total weight of the supported noble metal catalyst with a dopant.

10. The method according to claim 1, wherein the cleavage reaction is carried out in the presence of a zeolite having LTA, FAU, BEA, MFI or MOR framework.

11. The method according to claim 1, wherein the support of the supported noble metal catalyst is carbon.

12. The method according to claim 1, wherein the weight ratio of the compound comprising an aromatic ring and a C—O—C linkage to the catalyst is from 1:1 to 100:1 and preferably from 2:1 to 10:1.

13. The method according to claim 1, wherein the reaction temperature of the cleavage reaction is from 80 to 250° C.

14. The method according to claim 1, wherein the hydrogen source is H₂ and H₂ pressure is from 1 and 50 bars.

15. A mixture comprising:
i. a compound comprising an aromatic ring and a C—O—C linkage;
ii. a supported noble metal catalyst doped with a halogen selected from the group consisting of chlorine and bromine;
iii. a hydrogen source;
iv. optionally a solvent;
v. optionally a zeolite having LTA, FAU, BEA, MFI or MOR framework.
